SEQUENCE LISTING (17 sequences)

SEQ ID NO: 1 - DNA, 2747 bp, synthetic construct
  source 1..2747: mol_type = other DNA
atgctcgagggagtgcaggtggagactatctccccaggagacgggcgcaccttccccaagcgcggccagacctgcgtggtgcactacaccgggatgcttgaagtggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagtcgactatccgtacgacgtaccagactacgcactcgacggaagcggagccacgaacttctctctgttaaagcaagcaggagatgttgaagaaaaccccgggcctatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccttggccaacacttgtcactactttaacttatggtgttcaatgcttttcaagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataacgatcaccaggtgtacatcatggcagacaaacaaaagaatggaatcaaagctaacttcaaaattagacacaacattgaagatggaggcgttcaactagcagaccattatcaacaaaatactccaattggcgatgggcccgtccttttaccagacaaccattacctgtttacaacttctactctttcgaaagatcccaacgaaaagagagaccacatggtccttcttgagtttgtaacagctgctgggattacacatggcatggatgaactatacaaaggcagcagcggcagcagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 2 - DNA, 321 bp, synthetic construct
  source 1..321: mol_type = other DNA
ggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaa

SEQ ID NO: 3 - DNA, 846 bp, synthetic construct
  source 1..846: mol_type = other DNA
ggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatca

SEQ ID NO: 4 - DNA, 1239 bp, synthetic construct
  source 1..1239: mol_type = other DNA
atgctcgagggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagtcgactatccgtacgacgtaccagactacgcactcgac

SEQ ID NO: 5 - DNA, 711 bp, synthetic construct
  source 1..711: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 6 - DNA, 714 bp, synthetic construct
  source 1..714: mol_type = other DNA
atgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccttggccaacacttgtcactactttaacttatggtgttcaatgcttttcaagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataacgatcaccaggtgtacatcatggcagacaaacaaaagaatggaatcaaagctaacttcaaaattagacacaacattgaagatggaggcgttcaactagcagaccattatcaacaaaatactccaattggcgatgggcccgtccttttaccagacaaccattacctgtttacaacttctactctttcgaaagatcccaacgaaaagagagaccacatggtccttcttgagtttgtaacagctgctgggattacacatggcatggatgaactatacaaa

SEQ ID NO: 7 - DNA, 589 bp, Human betaherpesvirus 5
  source 1..589: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatc

SEQ ID NO: 8 - DNA, 511 bp, Mus musculus
  source 1..511: mol_type = other DNA
ttctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacct

SEQ ID NO: 9 - DNA, 18 bp, synthetic construct
  source 1..18: mol_type = other DNA
ggcagcagcggcagcagc

SEQ ID NO: 10 - DNA, 66 bp, synthetic construct
  source 1..66: mol_type = other DNA
ggaagcggagccacgaacttctctctgttaaagcaagcaggagatgttgaagaaaaccccgggcct

SEQ ID NO: 11 - protein, 107 aa, synthetic construct
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 12 - protein, 282 aa, synthetic construct
GFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 13 - protein, 413 aa, synthetic construct
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSVDYPYDVPDYALD

SEQ ID NO: 14 - protein, 236 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 15 - protein, 238 aa, synthetic construct
MSKGEELFTGVVPILVELDGDVNGHKFSVSGEGEGDATYGKLTLKFICTTGKLPVPWPTLVTTLTYGVQCFSRYPDHMKRHDFFKSAMPEGYVQERTIFFKDDGNYKTRAEVKFEGDTLVNRIELKGIDFKEDGNILGHKLEYNYNDHQVYIMADKQKNGIKANFKIRHNIEDGGVQLADHYQQNTPIGDGPVLLPDNHYLFTTSTLSKDPNEKRDHMVLLEFVTAAGITHGMDELYK

SEQ ID NO: 16 - protein, 6 aa, synthetic construct
GSSGSS

SEQ ID NO: 17 - protein, 22 aa, synthetic construct
GSGATNFSLLKQAGDVEENPGP

(19) United States
(12) Patent Application Publication
(10) Pub. No.: US 2025/0320523 A1
(43) Pub. Date: Oct. 16, 2025

(54) PHAGOCYTOSIS ASSAY COMBINING A SYNTHETIC CELL DEATH SWITCH AND A PHAGOCYTOSIS REPORTER SYSTEM

(71) Applicant: Hoffmann-La Roche Inc., Little Falls, NJ (US)

(72) Inventors: Colette Andrea BICHSEL, Sissach (CH); Gabrielle PY, Kingersheim (FR); Malte STEINBERG, Maisprach (CH); Roberto VILLASENOR SOLORIO, Binningen (CH); Florian WANKE, Basel (CH)

(73) Assignee: Hoffmann-La Roche Inc., Little Falls, NJ (US)

(21) Appl. No.: 19/074,072

(22) Filed: Mar. 7, 2025

Related U.S. Application Data
(63) Continuation of application No. PCT/EP2023/074822, filed on Sep. 11, 2023.

(30) Foreign Application Priority Data
Sep. 13, 2022 (EP) 22195247.6

(51) Int. Cl. C12N 15/86; C12N 9/64; G01N 21/77; G01N 33/50

(52) U.S. Cl. CPC C12N 15/86; C12N 9/6472; C12Y 304/22062; G01N 21/77; G01N 33/5005; C12N 2740/15043; G01N 2021/7786

(57) ABSTRACT

The present invention relates to a recombinant expression vector encoding an inducible cell death switch, a pH-stable fluorophore and a pH-sensitive fluorophore. Moreover, the invention relates to cells comprising said recombinant expression vector as well as their use in an in vitro phagocytosis assay.

### CROSS REFERENCE TO RELATED APPLICATIONS

This application is a continuation of International Application No. PCT/EP2023/074822, filed Sep. 11, 2023, which claims priority to European Patent Application No. 22195247.6, filed Sep. 13, 2022, each of which are incorporated herein by reference in its entirety.

### SEQUENCE LISTING

This application contains a Sequence Listing which has been submitted electronically in XML format and is hereby incorporated by reference in its entirety. Said XML copy, created on Feb. 26, 2025, is named “P37706-US_Seq_List.xml” and is 24 kilobytes in size.

### FIELD OF THE INVENTION

The present invention relates to a recombinant expression vector encoding an inducible cell death switch, a pH-stable fluorophore and a pH-sensitive fluorophore. Moreover, the invention relates to cells comprising said recombinant expression vector as well as their use in an in vitro phagocytosis assay.

### BACKGROUND OF THE INVENTION

Maintaining any organ in a functional state requires quick and efficient clearance of pathogens and cell debris. Macrophages as part of the immune system maintain tissue homeostasis by tethering, engulfing and digesting such particles by a process called phagocytosis. In the brain, microglia carry out the majority of apoptotic cell clearance. Macrophage and microglial dysfunction leads to inappropriate clearance and has been associated with multiple autoimmune and neurodegenerative diseases including Systemic Lupus Erythematosus, Alzheimer's and Parkinson's.

Given this important role of macrophages and microglia, various phagocytosis assays are currently used. Commonly used substrates are opsonized red blood cells, yeast particles, E. coli bioparticles, amyloid beta plaques, myelin or cell debris. The substrate is often labeled with the pH-sensitive dye pHrodo, which increases its fluorescence intensity upon a drop in pH that occurs in phagolysosomes, and thus indicates phagocytosis. The substrate is typically co-incubated with phagocytic cells for a fixed amount of time. The phagocytes are then isolated and the internalized substrate measured for example by ELISA (if the substrate has specific epitopes), in a plate reader (e.g. for RBCs) or by flow cytometry (e.g. for fluorescently labeled debris). Time-lapse imaging during co-incubation with the phagocytic cells is used to observe and quantify phagocytosis.

There are two major limitations of the current phagocytosis assays. The first is high experimental variability due to variable efficiencies in generating apoptotic cells and modifications of the apoptotic cells by labeling, leading to difficulties in quantifying and comparing results. The second is that there is no flexibility in timing. The assay starts when all components are mixed, which is not desirable in more complex multicellular in vitro models. We therefore identified the following needs for an improved phagocytosis assay.

### Need for Precise and Specific Generation of Apoptotic Cells

Cellular debris from apoptotic cells is among the most important substrates for phagocytosis, because cell clearance makes up a large portion of macrophage and microglial activity, both in physiological and pathological conditions. However, protocols to obtain apoptotic cells (for example, by incubating with staurosporine, a bacterial product) may result in variable proportions of dead cells. Another concern is that the product may contain other forms of cellular debris (e.g. necrotic cells, leaked DNA and other components).

In addition, when the substrate is labeled, its surface is modified (e.g. by adding a fluorescent dye), which may impact substrate recognition by the phagocyte.

To obtain a consistent and physiologically relevant apoptotic cell substrate, cellular apoptosis should ideally be induced with high efficiency, specificity, reproducibility and without subsequent modifications/external labeling.

### Need for Intrinsically Labeled Apoptotic Cells

Importantly, to clearly identify a phagocytic event, it is necessary to distinctly label phagocytosed and non-phagocytosed apoptotic cells. The commonly used pHrodo dyes emit weak fluorescence in non-phagocytosed cells and a strong signal in labeled cells that are taken up and are inside low-pH lysosomes upon phagocytosis. Due to variable labeling efficiencies and occasional detachment of the dye from the surface, the pHrodo signal is variable between experiments, and the threshold between high and low signal needs to be adjusted for each experiment.

### Need for On-Demand Apoptosis Gene Switch to Gain Assay Flexibility

Finally, in current assays, the apoptotic cells (or other substrates) need to be generated and labeled before coming in contact with the phagocytes. Thus, phagocytosis starts immediately upon mixture of the two components. However, for some complex assays (spheroids, organoids, organotypic structure models), multiple cell types need to be seeded together, often within a 3D matrix, and multiple days are needed for the cells to arrange and self-organize. Thus, if the phagocytosis assay needs to be carried out several days after seeding all components, the conventional assays cannot be used. In this case, it is necessary to have an apoptotic switch (on-system), where the phagocytosis substrate can be generated at the desired time point.

### Novelty and Advantages of the Present Invention Over Established Methods

The present inventors developed a novel phagocytosis assay which combines two features: 1) on-demand cell-specific apoptosis and 2) a phagocytosis specific fluorescent reporter. First, the inventors use a method where the apoptotic cells of any cell type can be generated on demand, with high efficiency and reproducibility, at any desired time point. The inventors make use of the inducible Caspase9 construct first described in Straathof, K C et al. An inducible caspase 9 safety switch for T-cell therapy. Blood. 2005; 105 (11): 4247-54 and disclosed in WO2011/146862 A1, wherein the dimerization domain of Caspase9 is replaced with the FKBP12-F36V dimerization domain. This substitution results in Caspase9 dimerization only upon addition of the small molecule AP20187, which subsequently triggers the apoptosis cascade.

Second, the inventors constitutively co-express in the same cell a fusion construct composed of mCherry and the GFP-based pH-sensitive Superecliptic pHluorin (SEP; Sankaranarayanan, S et al. The Use of pHluorins for Optical Measurements of Presynaptic Activity. Biophys. J. 2000; 79 (4): 2199-208). At pH 7.4, this reporter emits both green (SEP) and red (mCherry) fluorescence light upon excitation at 488 nm and 580 nm, respectively. In acidic pH, the SEP fluorescence is quenched and the reporter only emits red fluorescent light. With this genetically encoded reporter, we can distinguish phagocytosed cells from non-phagocytosed cells with high precision without externally modifying the cells. This novel construct, iCaspase9-SEP-mCherry (FIG. 1), can be transfected or transduced into mammalian cells (cell lines, primary cells, iPSC) and expressed transiently or stably.

The phagocytosis assay can be carried out in 2D or 3D, days after having seeded multiple cell types, including the inducible substrate cells (e.g. H4-iCaspase9-SEP-mCherry or Jurkat-iCaspase9-SEP-mCherry cells) and the phagocytic cells (e.g. iPSC-derived microglia or macrophages).

### SUMMARY OF THE INVENTION

In one aspect, the present invention provides a recombinant expression vector encoding an inducible cell death switch, a pH-stable fluorophore and a pH-sensitive fluorophore.

In one aspect, the recombinant expression vector is a viral vector. In one aspect, the viral vector is a lentiviral vector.

In one aspect, the inducible cell death switch induces apoptosis, necroptosis, pyroptosis or ferroptosis, preferably apoptosis. In another aspect, the inducible cell death switch comprises an inducer-binding domain and a signaling protein of a cell death pathway. In one aspect, the signaling protein of a cell death pathway is a pro-apoptotic protein, preferably Caspase9 or a functional fragment thereof. In one aspect, the inducer-binding domain comprises a dimerization domain, preferably FKBP12-F36V. In one aspect, the inducer-binding domain is capable of binding an inducer, preferably a chemical inducer of dimerization, most preferably AP20187. In one aspect, the inducible cell death switch is inducible Caspase 9.

In one aspect, the pH-stable fluorophore and the pH-sensitive fluorophore have distinct excitation and emission spectra. In a further aspect, the pH-stable fluorophore is from the RFP family, an Alexa Fluor dye, a protein-based fluorophore, a simple organic fluorophore or an organic polymer, preferably mCherry. In one aspect, the fluorescence of the pH-sensitive fluorophore changes in response to a decrease in pH, preferably wherein the fluorescence is quenched or the excitation/emission spectra are shifted. In another aspect, the pH-sensitive fluorophore is Superecliptic pHluorin (SEP), pHLemon, pHmScarlet, pHTomato, pHuji, a LysoSensor or a pH nanosensor, preferably Superecliptic pHluorin (SEP).

In one aspect, the recombinant expression vector comprises a promoter, particularly an ubiquitous promoter, a cell specific promoter, a constitutive promoter, or an inducible promoter. In one aspect, the recombinant expression vector comprises a CMV promoter. In one aspect, the recombinant expression vector comprises a polynucleotide sequence according to SEQ ID NO: 1.

According to a further aspect of the invention, a cell comprising the recombinant expression vector of the invention is provided. In one aspect, the cell is a mammalian cell, particularly a human cell. In one aspect, the cell is a stem cell. In a further aspect, the cell is a neural cell, neuronal cell, glial cell, mesenchymal cell or a haematopoietic cell, preferably a neuroglioma cell or a T cell. In one aspect, the cell is a H4 cell or a Jurkat cell. In one aspect, the expression of the recombinant expression vector in the cell is transient or stable.

Also encompassed by the invention is an in vitro method for evaluating phagocytosis, comprising the steps of: a) providing an inducible substrate cell according to the invention; b) combining and co-culturing the inducible substrate cell with phagocytic cells; c) inducing cell death of the inducible substrate cell; and d) detecting the fluorescent signals of the inducible substrate cell; wherein a change in fluorescent signal of the pH-sensitive fluorophore is indicative of phagocytosis of the inducible substrate cell.

In one aspect, the phagocytic cells of step b) are macrophages or tissue resident macrophages. In one aspect, the tissue resident macrophages are microglia.

In one aspect, the inducible substrate cell and the phagocytic cells are co-cultured in an in vitro model comprising additional cell types. In one aspect, the in vitro model is a 2D or 3D culture system. In another aspect, the in vitro model is an organ-on-a-chip, spheroid or organoid. In one aspect, the in vitro model is a neurovascular unit or blood-brain barrier spheroids.

In one aspect, cell death of the inducible substrate cell in step c) is induced after the inducible substrate cell is combined with the phagocytic cells in step b). In a further aspect, cell death of the inducible substrate cell in step c) is induced by an inducer, preferably a chemical inducer of dimerization, most preferably AP20187.

In another aspect, the fluorescent signals of step d) are detected by fluorescent imaging, flow cytometry or by a fluorescence plate reader. In one aspect, the fluorescent signal in step d) is detected at several time points after induction of cell death.

### BRIEF DESCRIPTION OF THE FIGURES

FIG. 1: Lentiviral vector map showing the iCaspase9 linked with a cleavable P2A linker to the SEP-mCherry fluorophores, placed under the control of a CMV promoter. The construct also contains a puromycin selection cassette under the control of an mPGK promoter.

FIG. 2: iCaspase9 transfected cells quickly undergo apoptosis when exposed to AP20187, whereas staurosporine only led to less apoptosis after 3 h incubation.

FIG. 3: H4-iCaspase-SEP-mCherry cells imaged in the green channel (top) and red channel (bottom). SEP is quenched at lower pH, whereas mCherry is present in all milieus tested.

FIG. 4: H4-iCaspase-SEP-mCherry cells co-cultured with labeled HMC3 microglia in a 2D phagocytosis assay. Apoptosis of the transduced H4 cells was triggered at to by adding 10 nM AP20187, and the cells were imaged for 8 h.

FIG. 5: Phagocytosis of H4-iCaspase-SEP-mCherry cells co-cultured with iPSC-derived microglia in a 2D, imaged on an Incucyte® device.

FIG. 6: Efferocytosis of Jurkat-iCaspase-SEP-mCherry cells by THP-1 macrophages, quantified by flow cytometry.

FIG. 7: Phagocytosis of H4-iCaspase-SEP-mCherry cells by HMC3 microglia in 3D blood-brain barrier (BBB) spheroids on day 2, after apoptosis was specifically induced in transduced H4 cells.

FIG. 8: Phagocytosis of H4-iCaspase-SEP-mCherry cells by HMC3 microglia in a 3D neurovascular unit model after 7 days of vessel self-assembly. Apoptosis was specifically induced in transduced H4 cells.

### DETAILED DESCRIPTION OF THE INVENTION

Terms are used herein as generally used in the art, unless otherwise defined in the following.

As used herein, the term “recombinant expression vector” refers to a polynucleotide molecule capable of directing the expression of polypeptides which are encoded therein by polynucleotide sequences. Recombinant expression vectors comprise regulatory sequences that lead to efficient transcription of the encoding polynucleotide sequences. In the context of the present invention, the term “recombinant expression vector encoding an inducible cell death switch, a pH-stable fluorophore and a pH-sensitive fluorophore” includes (i) a single vector encoding all of said elements (i.e. the inducible cell death switch, the pH-stable fluorophore and the pH-sensitive fluorophore), or (ii) a plurality of vectors each encoding one or more of said elements, and collectively encoding all of said elements. Accordingly, in some embodiments according to the invention, the inducible cell death switch, the pH-stable fluorophore and the pH-sensitive fluorophore are encoded by a single vector, while in other embodiments, the inducible cell death switch, the pH-stable fluorophore and the pH-sensitive fluorophore are encoded by a plurality of vectors.

As used herein, the term “inducible cell death switch” refers to a molecule that upon activation can elicit the death of cells expressing the molecule. The cell death switch comprises an inducer-binding domain and a signaling protein of a cell death pathway. The signaling protein is activated through an inducer, which is added to the environment of the cells and binds to the inducer-binding domain of the inducible cell death switch. Thereby cell death specifically of the cells expressing the inducible cell death switch is induced. In absence of the inducer, the cells expressing the inducible cell death switch show physiological rates of cell death, i.e. comparable to cells that do not express the inducible cell death switch. Thus, cell death can be induced at a specific time point.

As used herein, the term “pH-stable fluorophore” refers to a fluorescent protein that emits fluorescence independently of the pH of the environment in the sense that at various pH values the fluorophore emits fluorescence with the same fluorescent spectra. For example, subject to a decrease of pH in the environment the fluorescence of the fluorophore will be maintained. Hence, a pH-stable fluorophore expressed by a cell being phagocytized will maintain its fluorescence during the maturation of a phagolysosome, which is associated with a decrease in pH.

As used herein, the term “pH-sensitive fluorophore” refers to a fluorescent protein that emits a fluorescent signal dependent on the pH of the environment in the sense that the fluorescent emission changes at varying pH values. At varying pH values, the fluorophore may emit different fluorescent spectra or the fluorophore may exhibit increased or decreased fluorescence. A pH-sensitive fluorophore may at neutral pH emit a strong fluorescent signal and upon acidification of the environment the fluorescent signal may be quenched. For example, a pH-sensitive fluorophore expressed by a cell being phagocytized may exhibit a decrease in its fluorescence during the maturation of a phagolysosome, which is associated with a decrease in pH.

The term “promoter” as used herein is defined as it is generally understood by the skilled person, as a polynucleotide sequence of the recombinant expression vector that controls the expression of encoded polypeptides. The promoter recruits the transcriptional machinery of the cell to the expression vector and regulates when and/or where the encoded polypeptides are expressed. An expression vector may comprise several independent promoters that regulate expression of different polypeptides. Thus, different polypeptides may be under the control of different promoters, i.e. the expression of the polypeptides is regulated by separate promoters. The promoter may be a “constitutive promoter”, which is considered to give stable expression levels across varying conditions, or may be an “inducible promoter”, which drives expression in response to specific stimuli. Moreover, the promoter may be a “ubiquitous promoter”, which is active in a wide range of cell types and/or developmental stages, or may be a “cell-type specific promoter”, which is only active in one or more specific cell types.

The term “transfection” or “transfect” as used herein is defined as it is generally understood by the skilled person, as the process of introducing foreign polynucleotide sequences into cells by non-viral methods. Common transfection methods include calcium phosphate, cationic polymers (such as polyethylenimine (PEI)), magnetic beads, electroporation, and commercial lipid-based reagents such as Lipofectamine® and FuGENE®.

The term “transduction” or “transduce” as used herein is defined as it is generally understood by the skilled person, as the process of introducing foreign polynucleotide sequences into cells via a viral vector. Transduction in general results in the stable expression of the encoded polypeptides.

The term “in vitro model” as used herein refers to a cell culture system designed to replicate certain aspects of cellular behavior found in vivo, thereby facilitating the study of cellular processes. In vitro models may encompass a single cell type or may include two or more cell types as well as extracellular matrix and/or form-giving elements (e.g. a microfabricated device). Thereby, an in vitro model may be a simplified representation of different tissues or organs, or may mimic the in vivo structure and organization of tissues or organs. Thus, the cells of the in vitro model may be cultured in a 2D or a 3D culture system. A “2D culture system” refers to an in vitro model wherein the cells are cultured essentially as a monolayer (i.e. in a two dimensional structure). Whereas, a “3D culture system” refers to an in vitro model wherein the cells are organized in a three dimensional structure (e.g. an organ-on-a-chip, a spheroid or an organoid as described hereinbelow).

The term “organ-on-a-chip” as used herein refers to a culture system on a microfluidic chip that simulates the activities, mechanics and physiological responses of an organ or an organ system.

The term “spheroid” or “spheroid culture system” as used herein refers to a 3D in vitro model of cells grown in suspension, wherein cells aggregate and form a spheroid shape. Spheroids may provide a similar physicochemical environment to the cells as in vivo by facilitating cell-cell and cell-matrix interaction to overcome the limitations of traditional monolayer cell culture.

The term “organoid” as used herein refers to a 3D in vitro model that mimics its corresponding in vivo tissue or organ, such that it can be used to study aspects of that organ in the tissue culture dish.

The term “phagocytosis” as used herein refers to the cellular process of ingesting and eliminating particles, such as microorganisms, foreign substances, cells or cell debris. It encompasses the term “efferocytosis”, which refers to a specialized phagocytic process. During the phagocytic process, particles to be eliminated are engulfed by the cell membrane forming a specialized intracellular vacuole called a phagosome. The phagosome matures into a phagolysosome in which the engulfed particles are degraded and eliminated. Maturation of a phagosome into a phagolysosome is characterized by the acidification of the vacuole, i.e. a decrease in pH.

The term “phagocytosis assay” as used herein refers to a cellular assay wherein phagocytic activity is assessed. A phagocytosis assay in general includes at least one cell type which acts as phagocyte, and particles or substrates to be phagocytosed.

The term “phagocyte” or “phagocytic cell” as used herein refers to a cell type that shows phagocytic activity, i.e. is capable of phagocytosis.

The term “inducible substrate cell” as used herein refers to a cell comprising an inducible cell death switch, which after induction of cell death may be subject to phagocytosis, i.e. becomes a substrate for phagocytes.

The present invention provides a recombinant expression vector encoding an inducible cell death switch, a pH-stable fluorophore and a pH-sensitive fluorophore. The recombinant expression vector of the invention may be a viral vector. The recombinant expression vector may be a lentiviral vector, an adenoviral vector or a retroviral vector. In one embodiment, the recombinant expression vector is a lentiviral vector.

The inducible cell death switch encoded by the recombinant expression vector of the invention may comprise an inducer-binding domain and a signaling protein of a cell death pathway. Upon binding of an inducer, particularly a chemical inducer, to the inducer-binding domain, the signaling protein is activated which leads to the initiation of the cell death pathway. Activation of the signaling protein may be achieved by dimerization. Thus, the inducer-binding domain may be a dimerization domain. Upon binding of the inducer, the dimerization domain dimerizes with another dimerization domain, leading to the activation of the cell death signaling protein. The inducer-binding domain may be FKBP12-F36V as described in Straathof, K C et al. An inducible caspase 9 safety switch for T-cell therapy. Blood. 2005; 105 (11): 4247-54. FKBP12-F36V is a human FK506 binding protein (FKBP12; GenBank AH002 818) that comprises an F36V mutation. The polynucleotide sequence of FKBP12-F36V is shown in SEQ ID NO: 2. The amino acid sequence is shown in SEQ ID NO: 11. In one embodiment, the inducer-binding domain is a dimerization domain. In another embodiment, the inducer-binding domain is FKBP12-F36V. In one embodiment, the inducer-binding domain is encoded by the polynucleotide sequence of SEQ ID NO: 2. In one embodiment, the inducer-binding domain comprises the amino acid sequence of SEQ ID NO: 11. In one embodiment, the inducer is a chemical inducer. In a particular embodiment, the inducer is AP20187. In one embodiment, the inducer-binding domain is FKBP12-F36V, and the inducer is AP20187.

The signaling protein of a cell death pathway may amongst others be a signaling protein of the apoptosis pathway, necroptosis pathway, pyroptosis pathway or ferroptosis pathway. Thus, the inducible cell death switch may amongst others induce apoptosis, necroptosis, pyroptosis or ferroptosis. Preferably, the inducible cell death switch induces apoptosis. The signaling protein may be a pro-apoptotic protein such as a protein of the Caspase signaling cascade. Sequential activation of caspases plays a central role in the execution of cell apoptosis. The signaling protein may be Caspase9 (Casp9; UniProtKB: P55211) or a functional fragment thereof. The term “functional fragment” as used herein refers to a portion of a protein, which retains the biological function of the full-length protein, i.e. a functional fragment of a protein of a cell death pathway will also induce cell death. A functional fragment of Casspase9 may have the amino acid sequence as shown in SEQ ID NO: 12 or may be encoded by the SEQ ID NO: 3. In their monomeric form, Caspase9 or functional fragments of Caspase9 are inactive. Through dimerization, they are activated and function as an initiating caspase, activating downstream executioner caspases. In one embodiment, the signaling protein of a cell death pathway is Caspase9, particularly human Caspase9, or a functional fragment thereof. In one embodiment, the protein of a cell death pathway is encoded by the sequence of SEQ ID NO: 3. In one embodiment, the protein of a cell death pathway comprises the amino acid sequence of SEQ ID NO: 12.

As used herein, the term “inducible Caspase9” or “iCasp9” refers to the construct F-Casp9, also designated iCasp9M, as described by Straathof, K C et al., comprising FKBP12-F36V and a functional fragment of Caspase9. The amino acid sequence of inducible Caspase9 is shown in SEQ ID NO: 13. The polynucleotide sequence of inducible Caspase9 is shown in SEQ ID NO: 4. In one embodiment, the inducible cell death switch comprises a dimerization domain and a pro-apoptotic protein. In one embodiment, the inducible cell death switch comprises FKBP12-F36V and Caspase9. In one embodiment, the inducible cell death switch is inducible Caspase9. In one embodiment, the inducible cell death switch is encoded by the sequence of SEQ ID NO: 4. In one embodiment, inducible cell death switch comprises the sequence of SEQ ID NO: 13.

The recombinant expression vector of the invention encodes a pH-stable and a pH-sensitive fluorophore. The pH-stable fluorophore and the pH-sensitive fluorophore have distinct excitation and emission spectra, i.e. the fluorescent signals can be distinguished from each other by fluorescent detection systems. Thereby phagocytosed cells may be distinguished from non-phagocytosed cells in a phagocytosis assay. The pH-stable fluorophore may be mCherry, other fluorophores from the RFP family, Alexa Fluor dyes, protein-based fluorophores (e.g. PE), simple organic fluorophores or organic polymers. Generally, any pH-stable fluorophore that has a distinct excitation and emission spectrum from the pH-sensitive fluorophore can be used. mCherry is a member of the mFruits family of monomeric red fluorescent proteins (mRFPs). It absorbs light between 540-590 nm and emits light in the range of 550-650 nm. The polynucleotide sequence of mCherry is shown in SEQ ID NO: 5. The amino acid sequence of mCherry is shown in SEQ ID NO: 14. The pH-sensitive fluorophore may be Superecliptic pHluorin (SEP) or pHLemon in the green spectrum, pHmScarlet, pHTomato or pHuji in the red spectrum, a LysoSensor, a pH nanosensor (for example QD-protein FRET-based pH sensor) or a (molecular) fluorescent switch. SEP is a pH-sensitive green fluorescent protein that emits a strong green fluorescent signal at neutral pH. With acidification of the environment, the fluorescent signal progressively decreases, i.e. is quenched, with a pKa of 7.2 and an apparent Hill coefficient of 1.9. The polynucleotide sequence of SEP is shown in SEQ ID NO: 6. The amino acid sequence of SEP is shown in SEQ ID NO: 15. Alternatively, the excitation/emission spectra of the pH-sensitive fluorophore may change depending on the pH of the environment. Thus, during the phagocytic process the excitation/emission spectra of the pH-sensitive fluorophore may shift. In one embodiment, the pH-stable fluorophore is mCherry. In one embodiment, the pH-sensitive fluorophore is SEP. In one embodiment, the pH-stable fluorophore is mCherry and the pH-sensitive fluorophore is SEP. In one embodiment, the pH-stable fluorophore and the pH-sensitive fluorophore are linked via a linker thereby generating a fluorophore fusion protein. The linker may be encoded by the polynucleotide sequence of SEQ ID NO: 9. In one embodiment, mCherry and SEP are linked by the amino acid sequence of SEQ ID NO: 16.

The cell death switch and the fluorophore fusion protein may be linked by a cleavable P2A linker. In one embodiment, the cleavable P2A linker is encoded by the sequence of SEQ ID NO: 10. In one embodiment, iCaspase9 and SEP-mCherry fusion protein are linked by the amino acid sequence of SEQ ID NO: 17.

The inducible cell death switch, the pH-stable fluorophore and the pH-sensitive fluorophore encoded by the recombinant expression vector of the invention may be under the control of a promoter. Depending on the cell type to be transfected or transduced and/or the specific assay to be performed, the promoter may be a ubiquitous promoter, a cell-type specific promoter, a constitutive promoter, or an inducible promoter. For example, the immediate early gene of the human cytomegalovirus (CMV), also called CMV promoter, may be used. The sequence of the CMV promoter is shown in SEQ ID NO: 7. This promoter is considered to result in stable, constitutive expression of the encoded proteins in a wide range of cell types. Difficult to transfect or transduce cell types may benefit from a cell-type specific promoter. For example, for expression in neurons a neuron-specific promoter may be used. Furthermore, if undifferentiated stem cells are to be transduced or transfected with the recombinant expression vector, a cell-type specific promoter may be useful. Thereby, during or after differentiation the encoded proteins will only be expressed in a specific cell type. Further, an inducible promoter may be used to institute expression of the encoded proteins at a specific time point. In one embodiment, the cell death switch, the pH-stable and the pH-sensitive fluorophore are under the control of a promoter. In one embodiment, the expression vector comprises a CMV promoter. In one embodiment, the cell death switch, the pH-stable and the pH-sensitive fluorophore are under the control of a CMV promoter.

The recombinant expression vector of the invention may further comprise a selection marker gene. Selection marker genes are useful to select successfully transfected or transduced cells. The selection marker gene may be an antibiotic-resistance gene, for example the puromycin-resistance gene (Puro). In this case, the selection marker gene may be under the control of a promoter which is active in the transfected or transduced cell type. This promoter may be distinct from the promoter driving expression of the inducible cell death switch, the pH-stable fluorophore and the pH-sensitive fluorophore. The promoter may be mPGK (murine phosphoglycerate kinase), which is efficient in driving high expression in various cell types. The sequence of the mPGK promoter is shown in SEQ ID NO: 8. Alternatively, successfully transfected or transduced cells may be selected by fluorescence. For example, cells expressing the pH-stable fluorophore may be sorted by fluorescence-activated cell sorting (FACS). In one embodiment, the recombinant expression vector of the invention comprises a selection marker gene. In one embodiment, the recombinant expression vector of the invention comprises a puromycin-resistance gene. In one embodiment, the selection marker gene is under the control of a mPGK-promoter.

The recombinant expression vector of the invention may comprise antibiotic resistance genes to select viral vector-producing bacterial clones during vector production. In this case, the antibiotic-resistance gene is under the control of a bacterial promoter. In one embodiment, the recombinant expression vector comprises an antibiotic-resistance gene. In one embodiment, the recombinant expression vector comprises an ampicillin-resistance gene.

In one embodiment, the recombinant expression vector of the invention comprises a polynucleotide sequence according to SEQ ID NO: 1.

The invention further relates to a cell comprising the recombinant expression vector of the invention. Since the cell death of such a cell can be initiated at a specific time point and its phagocytic uptake visualized by the expressed fluorophores, the cell is especially suitable as an inducible substrate cell in a phagocytosis assay.

In principle, the recombinant expression vector of the invention may be expressed in any cell type. Thus, the cell comprising the recombinant expression vector of the invention may be of any cell type. The cell may be a cell line, derived from induced pluripotent stem cells (iPSC-derived), derived from embryonic stem cells (ESC-derived) or patient-derived. The cell may be for example a neural cell, neuronal cell, glial cell, mesenchymal cell or a haematopoietic cell. The cell may be a healthy or a diseased cell. A diseased cell may be for example a cancer cell, such as a neuroglioma cell. Thereby, for example the phagocytosis of apoptotic neuroglioma cells, e.g. by microglia, may be evaluated.

The cell type may be chosen depending on the phagocytosis assay to be performed. For example, if the phagocytosis of leukemic T cells is to be studied, the cell comprising the recombinant expression vector of the invention may be a T cell.

Moreover, the cell comprising the recombinant expression vector of the invention may be a stem cell. By selecting a cell-type specific promoter that controls the expression of the encoded polypeptides, only specific cell types will express the encoded polypeptides after differentiation. In one embodiment, the cell comprising the recombinant expression vector of the invention is a mammalian cell, particularly a human cell. In one embodiment, the cell comprising the recombinant expression vector of the invention is a stem cell. In one embodiment, the cell comprising the recombinant expression vector of the invention is a neuroglioma cell or T cell. In one embodiment, the cell comprising the recombinant expression vector of the invention is a H4 cell or a Jurkat cell.

The cell may be transfected or transduced with the recombinant expression vector of the invention. Thus, the expression of the encoded polypeptides may be transient or stable. In one embodiment, the expression of the recombinant expression vector in the cell is stable. In a further embodiment, the expression of the recombinant expression vector of the invention in the cell is transient.

The invention further relates to an in vitro method for evaluating phagocytosis. The in vitro method described herein provides an improved method of generating substrate cells and assessing their phagocytosis. Thereby, substrate cells according to the invention are co-cultured with phagocytes, the cell death of the substrate cells is induced and their phagocytosis by the phagocytes is assessed. The substrate cell expresses both a pH-stable and a pH-sensitive fluorophore. Since phagocytosis is associated with a decrease in pH in the phagolysosome, a change in fluorescent signal of the pH-sensitive fluorophore indicates the phagocytosis of a substrate cell. The pH-stable fluorophore enables precise localization of the phagocytic event.

In one embodiment, the in vitro method for evaluating phagocytosis comprises the steps of: a) providing inducible substrate cells according to the invention as disclosed herein (i.e. cells comprising the recombinant expression vector of the invention as described hereinabove); b) combining and co-culturing the inducible substrate cells with phagocytic cells; c) inducing cell death of the inducible substrate cells; and d) detecting the fluorescent signals of the inducible substrate cells; wherein a change in fluorescent signal of the pH-sensitive fluorophore is indicative of phagocytosis of the inducible substrate cell.

It is understood that any of the steps b) to d) may coincide in time or may be performed sequentially, in any order. For example, cell death of the substrate cells may be induced at the same time as the substrate cells and the phagocytes are combined, i.e. steps b) and c) may coincide. Alternatively, the cell death of the substrate cells may be induced before or after the cells have been combined. Likewise, the fluorescent signals may be detected at any time points.

The phagocytic cells may be macrophages or tissue resident macrophages. Tissue resident macrophages may be microglia. As macrophages and microglia arise from the same progenitor cells, their transcriptome and proteome overlap. In vitro, macrophages can be derived by differentiating macrophage/microglia precursor cells using M-CSF or GM-CSF for seven days. Differentiation of microglial cells from macrophage/microglia precursor cells can be induced by M-CSF, TGF-beta1, and IL-34. Microglia markers CX3CR1, P2RY12 or TMEM119 can be used to distinguish them from each other.

The inducible substrate cells and the phagocytes may be co-cultured in an in vitro model comprising additional cell types. Thereby, the in vitro method allows for the evaluation of phagocytosis in different types of in vitro models mimicking different tissues or organs. Thus, depending on the in vitro model to be studied, various additional cell types may be employed. For example, studying the phagocytosis of neuroblastoma cells by microglia, the in vitro model may comprise brain endothelial cells, pericytes and astrocytes as additional cell types. In one embodiment, the inducible substrate cells and the phagocytes are co-cultured in an in vitro model comprising additional cell types.

An advantage of the in vitro method described herein is that the phagocytosis of the substrate cells does not necessarily commence at the specific time point when substrate cells and phagocytes are combined in an in vitro model. This allows the evaluation of phagocytosis in in vitro models that initially require the different cell types to organize (e.g. differentiate, migrate, adhere, connect), i.e. establish the in vitro model, before the phagocytosis assay is initiated. Thus, the cell death of the inducible substrate cells and their phagocytosis may be induced after a period of time wherein the in vitro model is established. This is especially the case for 3D in vitro models, such as organs-on-a-chip, spheroids or organoids. These culture systems may require several cell seeding steps and/or a certain period of time for the cells to organize.

It is further envisioned that the in vitro method of evaluating phagocytosis may be performed in in vitro models that have been established through differentiation of stem cells. The recombinant expression vector of the invention, under the control of a cell-type specific promoter, may be transduced into undifferentiated stem cells. After differentiation and the establishment of the stem cell derived in vitro model, the cell death switch, the pH-stable and the pH-sensitive fluorophore would be expressed solely in the cell type wherein the cell-type specific promoter is active. This enables the study of phagocytosis in stem cell derived culture systems.

Thus, in one embodiment, the cell death of the inducible substrate cells is induced after a period of time wherein the cells establish the in vitro model. In one embodiment, the cell death of the inducible substrate cells is induced after the inducible substrate cells are combined with the phagocytic cells (i.e. steps b and c of the in vitro method of evaluating phagocytosis). In further embodiments, the cell death of the inducible substrate cells is induced after about 1 hour, after about 2 hours, after about 3 hours, after about 4 hours, after about 5 hours or after about 6 hours of combining and co-culturing the inducible substrate cells and the phagocytes. In further embodiments, the cell death of the inducible substrate cells is induced after about 1 day, after about 2 days, after about 3 days, after about 4 days, after about 5 days, after about 6 days or after about 7 days of combining and co-culturing the inducible substrate cells and the phagocytes.

As described above, the cell death of the inducible substrate cell may be induced at any specific time point chosen depending on the in vitro model used or on the preference of the person performing the in vitro method. Cell death is induced by the presence of an inducer, e.g. a chemical inducer, in the environment of the inducible substrate cell. Thus, the inducer may be added to the cell culture media at any chosen time point. The inducer binds to the inducer-binding domain of the cell death switch and activates the signaling protein of a cell death pathway. The activated signaling protein initiates the respective cell death pathway within the inducible substrate cell, leading to its cell death. Depending on the cell death switch and its inducer-binding domain employed in the inducible substrate cells, different inducers may be used. The inducer-binding domain may be a dimerization domain. Therefore, the inducer may be an inducer of dimerization. The inducible cell death switch may comprise FKBP12-F36V as dimerization domain. Thus, the chemical inducer of dimerization may be AP20187 (Formula I).

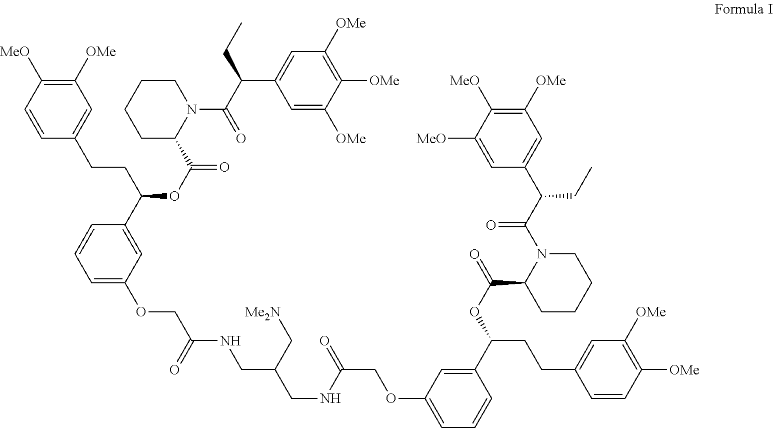 [SMILES: CC[C@H](C(=O)N1CCCC[C@H]1C(=O)O[C@H](CCc1ccc(OC)c(OC)c1)c1cccc(OCC(=O)NCC(CNC(=O)COc2cccc([C@@H](CCc3ccc(OC)c(OC)c3)OC(=O)[C@@H]3CCCCN3C(=O)[C@@H](CC)c3cc(OC)c(OC)c(OC)c3)c2)CN(C)C)c1)c1cc(OC)c(OC)c(OC)c1]

AP20187 (CAS No.: 195514-80-8) is a nontoxic synthetic FK506 analog that has been modified to reduce interactions with endogenous FKBPs, while enhancing binding to this FKBP12-F36V.

Binding of AP20187 to the inducer-binding domain of inducible Caspase9 causes dimerization and activation of Caspase9, leading to the initiation of apoptosis. In one embodiment, the cell death of the inducible substrate cells in step c) is induced by a chemical inducer of dimerization. In one embodiment, the cell death of the inducible substrate cells in step c) is induced by AP20187. In one embodiment, the cell death of the inducible substrate cells in step c) is induced by 10 nM AP20187.

As described herein, the change in fluorescent signal of the pH-sensitive fluorophore indicates the phagocytosis of an inducible substrate cell, while the pH-stable fluorophore enables localization of the cell. The fluorescent signals of an inducible substrate cell may be detected by fluorescent imaging, flow cytometry or a fluorescence plate reader.

The fluorescent signal of the pH-stable and/or the pH-sensitive fluorophore of the substrate cells may be detected at any time point of the in vitro method described herein. The fluorescent signals may be detected before and after cell death of the substrate cells is induced. Furthermore, the fluorescent signals may be detected at several time points during the phagocytosis assay. Thereby a time course study of phagocytosis may be performed. In one embodiment, the fluorescent signals of the pH-stable and/or the pH-sensitive fluorophore are detected before induction of cell death of the substrate cells. In a further embodiment, the fluorescent signals of the pH-stable and/or the pH-sensitive fluorophore are detected at several time points after induction of cell death of the substrate cells. In one embodiment, the fluorescent signals of the pH-stable and/or the pH-sensitive fluorophore are detected every 5 minutes, every 10 minutes, every 15 minutes, every 20 minutes or every 30 minutes after induction of cell death. In a further embodiment, the fluorescent signals of the pH-stable and/or the pH-sensitive fluorophore are detected after about 1 hour, after about 2 hours, after about 3 hours, after about 4 hours, after about 5 hours, after about 6 hours, after about 7 hours and/or after about 8 hours of induction of cell death.

### EXAMPLES

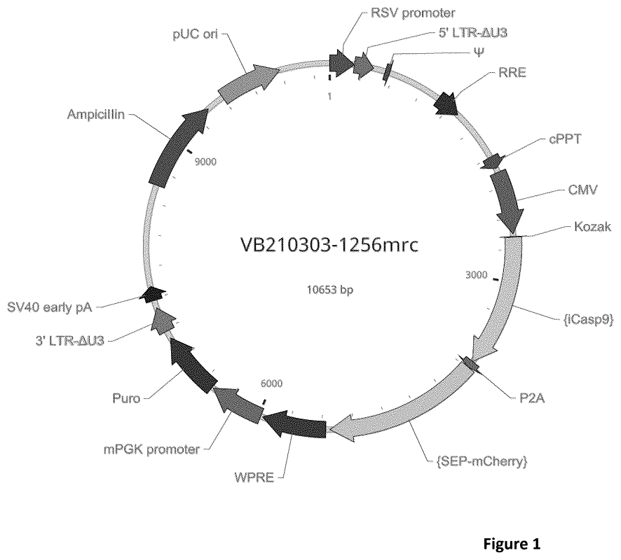

The iCaspase9-SEP-mCherry construct (SEQ ID NO: 1; FIG. 1) was transduced in the cell lines H4 (neuroblastoma) and Jurkat (T lymphocytes), and HEK293 were transfected with the iCaspase9 construct. H4 and Jurkat clones with a stable expression of the construct were selected. As phagocytes the cell line HMC3 (microglia) or iPSC-derived macrophages or microglia (produced in-house as described in WO2020/239714 A1) were used.

### Example 1: Efficient On-Demand Generation of Apoptotic Cells

HEK293 cells were transfected with 2 μg of iCaspase9 construct by nucleofection (Amaxa 4D nucleofector by Lonza, program CM-130) and plated in a 24-well plate. The following day, 100 nM AP20187 was added to the cells, and analyzed 1 h, 2 h or 3 h later. For the control, the cells were incubated with 2.5 M Staurosporine for 3 h. For the analysis, the cells were harvested and labeled with AnnexinV-BV421 and viability dye APCeF780 and analyzed by flow cytometry. Apoptotic cells were qualified as AnnexinV-positive and viability dye-negative.

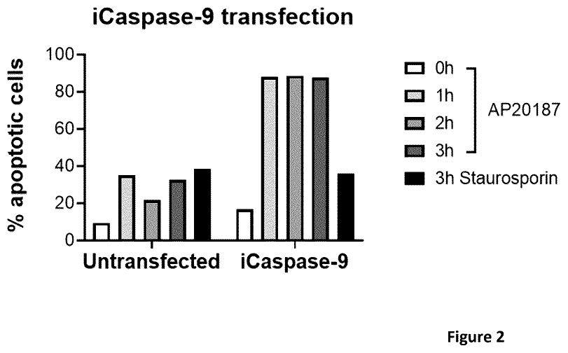

The HEK293 cells transfected with iCaspase9 undergo rapid and sustained apoptosis upon treatment with 100 nM AP20187. After 1 h, 88% of iCaspase9-transduced cells were apoptotic, as compared to 36% apoptotic cells after 3 h of Staurosporine treatment (FIG. 2). Untransfected cells showed basal apoptosis levels between 10-35%.

In conclusion, inducing apoptosis with AP20187 in cells expressing iCaspase9 is faster and more efficient than the standard method of inducing apoptosis with Staurosporine.

### Example 2: Precise Quantification of Phagocytosis with Increased Dynamic Range

2.1. pH-Sensitive Dual Fluorescent Indicator

H4 cells were transduced with a lentiviral vector containing iCaspase-SEP-mCherry and expanded in 1 μg/ml Puromycin-containing media to select for transduced cells. The cells were placed in different pH solutions (Intracellular pH Calibration Buffer Kit, invitrogen) and imaged on an inverted fluorescence microscope (Leica).

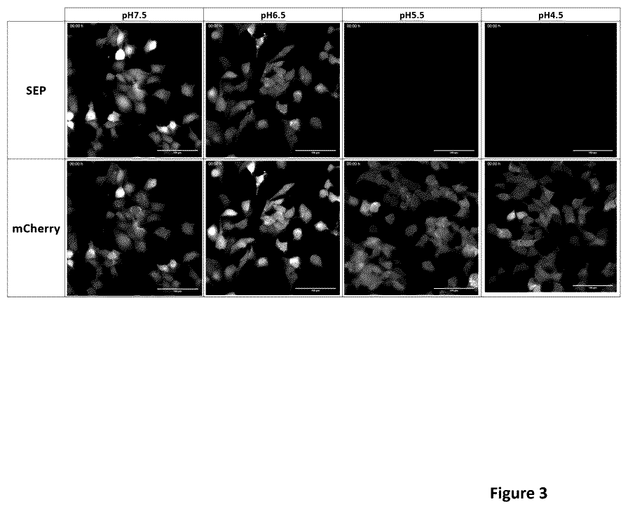

At physiological pH of 7.5, mCherry and SEP are co-expressed and co-localize in the transduced cells (FIG. 3). The mCherry fluorescence is present irrespective of the pH (FIG. 3, lower panel), whereas the SEP fluorescent signal is strong at physiological pH 7.4 but quenched in acidic conditions (FIG. 3, upper panel).

H4-iCaspase-SEP-mCherry express mCherry fused to SEP constitutively, and show quenching of the SEP fluorophore in acidic milieus. Therefore, this construct is a suitable fluorescent pH-sensor.

### 2.2. Assessment of Phagocytosis of H4 Cells by Microglia in 2D Model

H4 cells were transduced with a lentiviral vector containing iCaspase-SEP-mCherry, single-cell sorted for SEP and mCherry double-positive cells and expanded in 1 μg/ml Puromycin-containing media to select for stably transduced cells. HMC3 or iPSC-derived microglia (Reich, M et al. Alzheimer's Risk Gene TREM2 Determines Functional Properties of New Type of Human iPSC-Derived Microglia. 2021; 11:617860) were seeded in a 96-well imaging plate and. To visualize microglial morphology during the phagocytosis assay, cells were stained with a far-red viability dye (eBioscience). H4-iCaspase-SEP-mCherry cells were added the next day and left to attach for 3 h. For control wells, 1 uM Cytochalasin D (a fungal toxin that binds to actin filaments and thereby inhibits actin polymerization/phagocytosis) was added 30 min before starting the time-lapse imaging. Apoptosis was induced at to by adding 10 nM AP20187 to the media, and time-lapse imaging was immediately started on an inverted fluorescence microscope (Leica) or incucyte (Sartorius), 10× objective, 1 image every 10 minutes for 8 hours or more.

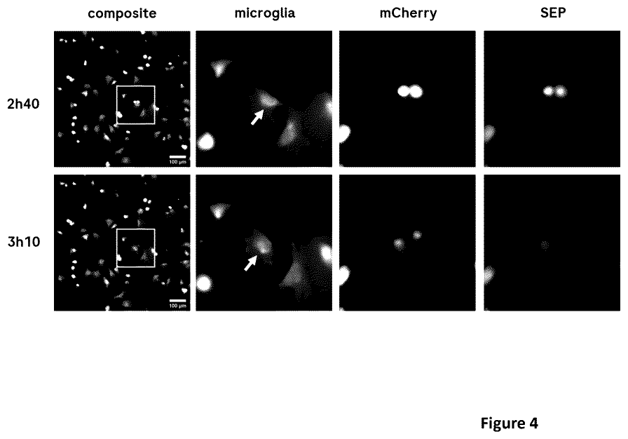

Apoptotic H4-iCaspase-SEP-mCherry were actively phagocytosed by co-cultured HMC3 microglia (FIG. 4). The mCherry signal designates transduced H4 cells and the SEP signal allows to determine the precise time point of phagocytosis by microglia (loss in SEP signal indicates phagocytosis). The example highlighted in FIG. 4 shows at 2 h40 after apoptosis initiation (the upper row) co-localization of a microglia (arrow) with an apoptotic H4 cell, and at 3 h10 after apoptosis initiation (the lower row) phagocytosis of the H4 cell, as evidenced by the loss of SEP signal.

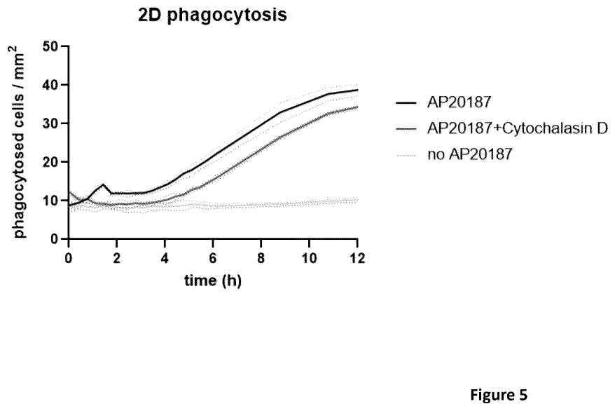

FIG. 5 shows phagocytosis of apoptotic H4-iCaspase-SEP-mCherry by iPSC-derived microglia. The number of phagocytosed cells per area was determined using the incucyte software for automated image quantification, by 1) segmenting individual cells, 2) identifying all H4-iCaspase-SEP-mCherry cells positive for mCherry (red fluorescence) and 3) from those cells, identifying the SEP (green fluorescence) negative cells as phagocytosed cells. Apoptotic cells are phagocytosed by iPSC-derived microglia over the course of 12 hours, when a plateau is reached.

Adding Cytochalasin D reduces both the onset and total amount of phagocytosed apoptotic cells, and without inducing apoptosis no phagocytosis is observed (FIG. 5).

The fast and efficient induction of apoptosis leads to a fast phagocytosis that can be precisely and reproducibly quantified.

### 2.3. Assessment of Efferocytosis of Jurkat Cells by Macrophages in 2D Model

Jurkat cells were transduced with a lentiviral vector containing iCaspase-SEP-mCherry, FACS sorted for SEP- and mCherry-expression and expanded in 1 ug/ml Puromycin-containing media to select for stably transduced cells. For pre-treatments, the Jurkat cells were incubated with GAS6, an efferocytosis enhancer, or Cytochalasin D, an efferocytosis inhibitor, then exposed to 10 nM AP20187 and incubated with THP-1 macrophages. As a control, Jurkat cells were treated with Staurosporin and labeled with pHrodo (red pH-sensitive fluorophore). Efferocytosis was quantified using flow cytometry, by gating for mCherry-positive and SEP-negative cells (apoptotic cells taken up by THP1), and for pHrodo-high cells in the control samples.

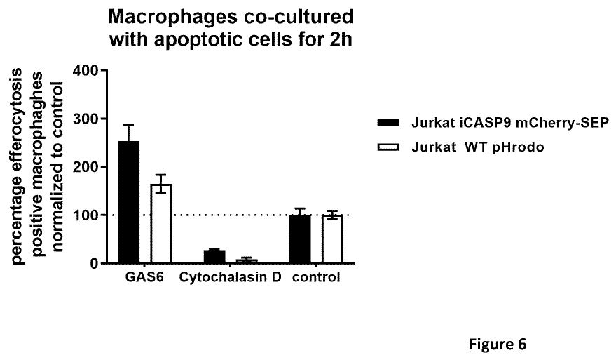

The range between highest uptake (GAS6-pre-treated) and lowest uptake (Cytochalasin D pre-treated) of apoptotic cells by macrophages is 2-3 times higher using the iCaspase9-SEP-mCherry construct than the Staurosporin treated and pHrodo-labeled Jurkat cells (FIG. 6). This increased range may be due to reduced fluorescence background and/or increased efferocytosis.

iCaspase-SEP-mCherry transduced apoptotic cells induce stronger phagocytosis/efferocytosis compared to conventional substrates, and thus give a higher dynamic range. This allows for a better ranking of different modulators of phagocytosis, adding value to this assay compared to the conventional approach.

### Example 3: Application of New Phagocytosis Assay to Complex 3D Co-Culture Models

### 3.1. Use of Novel Phagocytosis Assay in 3D BBB Spheriods

H4 cells were transduced with a lentiviral vector containing iCaspase-SEP-mCherry, single-cell sorted for SEP- and mCherry double-positive cells and expanded in 1 μg/ml Puromycin-containing media to select for stably transduced cells. H4-iCaspase-SEP-mCherry and HMC3 microglia were co-seeded with primary human brain endothelial cells, pericytes and astrocytes (Sciencell) to form blood-brain barrier (BBB) spheroids as described by Simonneau, C et al. Investigating receptor-mediated antibody transcytosis using blood-brain barrier organoid arrays. Fluids Barriers CNS. 2021; 18 (1): 43. Briefly, the cells were combined at a 1:1:1:1:1 ratio and seeded into a glass-bottom GRI3D microwell plate (SUN Biosciences). After 2 days of culture in EGM2 medium (Lonza) with astrocyte growth supplement (Sciencell), a timelapse experiment was performed as described above (for FIG. 4): Apoptosis was induced at t0 by adding 100 nM AP20187 to the media, and time-lapse imaging was immediately started on an inverted fluorescence microscope (Leica) using autofocus, 10× objective, 1 image every 10 minutes for 8 hours or more.

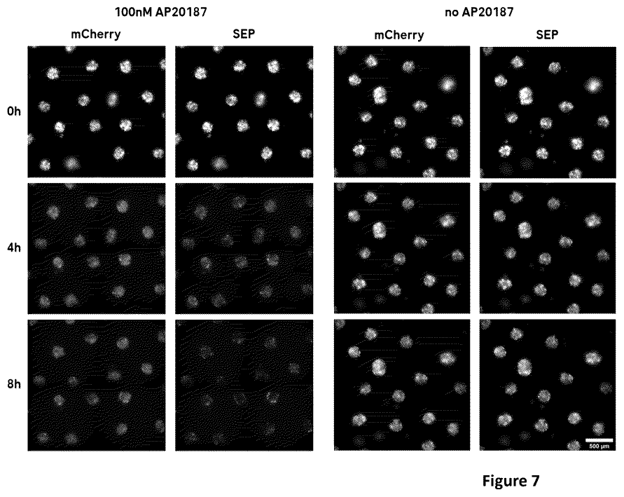

Spheroids containing H4-iCaspase-SEP-mCherry gradually lost SEP signal upon AP20187 treatment, indicating active phagocytosis within the spheroid cores (FIG. 7). SEP and mCherry fluorescence remained stable over the period of 8 hours in untreated spheroids.

iCaspase9-SEP-mCherry transduced H4 cells were successfully integrated in a high-throughput 3D in vitro model. Apoptosis and subsequent phagocytosis was triggered after 2 days, when the 3D architecture of the complex cellular model was established. It is not possible to compare this assay to other phagocytosis assays, because the substrate generation (apoptotic cells) cannot be triggered in a cell-specific manner after cell seeding in other known assays.

### 3.2. Use of Novel Phagocytosis Assay in a 3D Neurovascular Unit Model.

H4 cells were transduced with a lentiviral vector containing iCaspase-SEP-mCherry, single-cell sorted for SEP- and mCherry double positive cells and expanded in 1 μg/ml Puromycin-containing media to select for stably transduced cells. H4-iCaspase-SEP-mCherry and HMC3 microglia were co-seeded with primary human brain endothelial cells, pericytes and astrocytes (Sciencell) at the ratio 1:1:4:0.5:1 and left to self-assemble to neurovascular units inside a fibrin gel matrix in a microfluidic chip (AIM Biotech). The cells were seeded into the microfluidic chips as described in Campisi, M et al.3D Self-Organized Human Blood-Brain Barrier in a Microfluidic Chip. Methods Mol Biol. 2021; 2258:205-219, and the vascular networks were grown over 7 days in EGM2 medium (Lonza) with astrocyte growth supplement (Sciencell). Apoptosis was induced at t0 by adding 10 nM AP20187 to the media, the chips were then fixed in 4% PFA after 30 minutes or 2 h, counterstained with DAPI and imaged on an inverted fluorescence microscope.

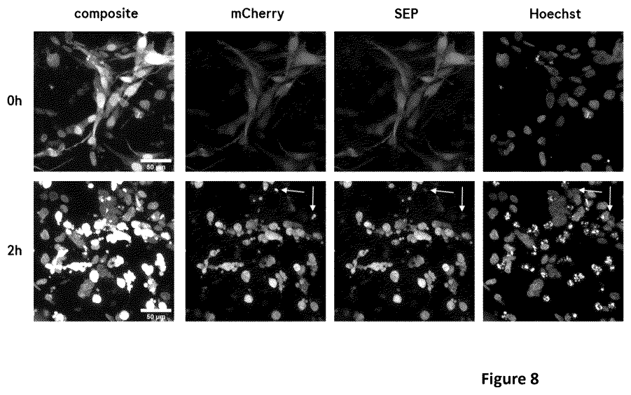

H4-iCaspase-SEP-mCherry cells surrounded vessel structures after 7 days in culture. Apoptosis was efficiently induced in the transduced cells within 30 minutes inside the 3D matrix of the microfluidic chip. The single-cell resolution in this setup allows to identify single instances of SEP signal loss in apoptotic cells after 2 h (FIG. 8), indicating local acidification due to phagocytosis.

iCaspase9-SEP-mCherry transduced H4 cells were successfully integrated in a complex 3D in vitro model. Apoptosis and subsequent phagocytosis was triggered after 7 days in culture. Single events of phagocytosis were observed. The system would further allow the co-localization of the phagocytosis events to individual microglia. Other phagocytosis assays do not have the option to trigger substrate generation (apoptotic cells) on demand and in a cell-specific manner, so we cannot compare our results to other phagocytosis assays.

### SEQUENCES

Lentiviral vector sequence iCaspase9-SEP-mCherry (SEQ ID NO: 1):

| | | |
|---|---|---|
| {iCasp9} | 2578-3816 | 1239 ORF |
| P2A | 3817-3882 | 66 Linker |
| {SEP-mCherry} | 3883-5325 | 1443 ORF, GSSGSS linker between SEP and mCherry |

ATG CTCGAGGGAG TGCAGGTGGA GACTATCTCC CCAGGAGACG GGCGCACCTT
CCCCAAGCGC GGCCAGACCT GCGTGGTGCA CTACACCGGG ATGCTTGAAG
TGGAAAGAA AGTTGATTCC TCCCGGGACA GAAACAAGCC CTTTAAGTTT
ATGCTAGGCA AGCAGGAGGT GATCCGAGGC TGGGAAGAAG GGGTTGCCCA
GATGAGTGTG GGTCAGAGAG CCAAACTGAC TATATCTCCA GATTATGCCT
ATGGTGCCAC TGGGCACCCA GGCATCATCC CACCACATGC CACTCTCGTC
TTCGATGTGG AGCTTCTAAA ACTGGAATCT GGCGGTGGAT CCGGAGTCGA
CGGATTTGGT GATGTCGGTG CTCTTGAGAG TTTGAGGGGA AATGCAGATT
TGGCTTACAT CCTGAGCATG GAGCCCTGTG GCCACTGCCT CATTATCAAC
AATGTGAACT TCTGCCGTGA GTCCGGGCTC CGCACCCGCA CTGGCTCCAA
CATCGACTGT GAGAAGTTGC GGCGTCGCTT CTCCTCGCTG CATTTCATGG
TGGAGGTGAA GGGCGACCTG ACTGCCAAGA AAATGGTGCT GGCTTTGCTG
GAGCTGGCGC GGCAGGACCA CGGTGCTCTG GACTGCTGCG TGGTGGTCAT
TCTCTCTCAC GGCTGTCAGG CCAGCCACCT GCAGTTCCCA GGGGCTGTCT
ACGGCACAGA TGGATGCCCT GTGTCGGTCG AGAAGATTGT GAACATCTTC
AATGGGACCA GCTGCCCCAG CCTGGGAGGG AAGCCCAAGC TCTTTTTCAT
CCAGGCCTGT GGTGGGGAGC AGAAAGACCA TGGGTTTGAG GTGGCCTCCA
CTTCCCCTGA AGACGAGTCC CCTGGCAGTA ACCCCGAGCC AGATGCCACC
CCGTTCCAGG AAGGTTTGAG GACCTTCGAC CAGCTGGACG CCATATCTAG
TTTGCCCACA CCCAGTGACA TCTTTGTGTC CTACTCTACT TTCCCAGGTT
TTGTTTCCTG GAGGGACCCC AAGAGTGGCT CCTGGTACGT TGAGACCCTG
GACGACATCT TTGAGCAGTG GGCTCACTCT GAAGACCTGC AGTCCCTCCT
GCTTAGGGTC GCTAATGCTG TTTCGGTGAA AGGGATTTAT AAACAGATGC
CTGGTTGCTT TAATTTCCTC CGGAAAAAAC TTTTCTTTAA AACATCAGTC
GACTATCCGT ACGACGTACC AGACTACGCA CTCGACGGAA GCGGAGCCAC
GAACTTCTCT CTGTTAAAGC AAGCAGGAGA TGTTGAAGAA AACCCCGGGC
CTATGAGTAA AGGAGAAGAA CTTTTCACTG GAGTTGTCCC AATTCTTGTT
GAATTAGATG GTGATGTTAA TGGGCACAAA TTTTCTGTCA GTGGAGAGGG
TGAAGGTGAT GCAACATACG GAAAACTTAC CCTTAAATTT ATTTGCACTA
CTGGAAAACT ACCTGTTCCT TGGCCAACAC TTGTCACTAC TTTAACTTAT
GGTGTTCAAT GCTTTTCAAG ATACCCAGAT CATATGAAAC GGCATGACTT
TTTCAAGAGT GCCATGCCCG AAGGTTATGT ACAGGAAAGA ACTATATTTT
TCAAAGATGA CGGGAACTAC AAGACACGTG CTGAAGTCAA GTTTGAAGGT
GATACCCTTG TTAATAGAAT CGAGTTAAAA GGTATTGATT TTAAAGAAGA
TGGAAACATT CTTGGACACA AATTGGAATA CAACTATAAC GATCACCAGG
TGTACATCAT GGCAGACAAA CAAAAGAATG GAATCAAAGC TAACTTCAAA
ATTAGACACA ACATTGAAGA TGGAGGCGTT CAACTAGCAG ACCATTATCA
ACAAAATACT CCAATTGGCG ATGGGCCCGT CCTTTTACCA GACAACCATT
ACCTGTTTAC AACTTCTACT CTTTCGAAAG ATCCCAACGA AAAGAGAGAC
CACATGGTCC TTCTTGAGTT TGTAACAGCT GCTGGGATTA CACATGGCAT
GGATGAACTA TACAAAGGCA GCAGCGGCAG CAGCATGGTG AGCAAGGGCG
AGGAGGATAA CATGGCCATC ATCAAGGAGT TCATGCGCTT CAAGGTGCAC
ATGGAGGGCT CCGTGAACGG CCACGAGTTC GAGATCGAGG GCGAGGGCGA
GGGCCGCCCC TACGAGGGCA CCCAGACCGC CAAGCTGAAG GTGACCAAGG
GTGGCCCCCT GCCCTTCGCC TGGGACATCC TGTCCCCTCA GTTCATGTAC
GGCTCCAAGG CCTACGTGAA GCACCCCGCC GACATCCCCG ACTACTTGAA
GCTGTCCTTC CCCGAGGGCT TCAAGTGGGA GCGCGTGATG AACTTCGAGG
ACGGCGGCGT GGTGACCGTG ACCCAGGACT CCTCCCTGCA GGACGGCGAG
TTCATCTACA AGGTGAAGCT GCGCGGCACC AACTTCCCCT CCGACGGCCC
CGTAATGCAG AAGAAGACCA TGGGCTGGGA GGCCTCCTCC GAGCGGATGT
ACCCCGAGGA CGGCGCCCTG AAGGGCGAGA TCAAGCAGAG GCTGAAGCTG
AAGGACGGCG GCCACTACGA CGCTGAGGTC AAGACCACCT ACAAGGCCAA
GAAGCCCGTG CAGCTGCCCG GCGCCTACAA CGTCAACATC AAGTTGGACA
TCACCTCCCA CAACGAGGAC TACACCATCG TGGAACAGTA CGAACGCGCC
GAGGGCCGCC ACTCCACCGG CGGCATGGAC GAGCTGTACA AGTAA
FKBP12-F36V sequence (SEQ ID NO: 2):
GGAGTGCAGGTGGAGACTATCTCCCCAGGAGACGGGCGCACCTTCCCCAAGCGCGG
CCAGACCTGCGTGGTGCACTACACCGGGATGCTTGAAGATGGAAAGAAAGTTGATT
CCTCCCGGGACAGAAACAAGCCCTTTAAGTTTATGCTAGGCAAGCAGGAGGTGATC
CGAGGCTGGGAAGAAGGGGTTGCCCAGATGAGTGTGGGTCAGAGAGCCAAACTGAC
TATATCTCCAGATTATGCCTATGGTGCCACTGGGCACCCAGGCATCATCCCACCACA
TGCCACTCTCGTCTTCGATGTGGAGCTTCTAAAACTGGAA
Truncated Caspase9 sequence (AA135-416 from Homo sapiens Caspase9, transcript variant
alpha NM_001229.5) (SEQ ID NO: 3):
GGATTTGGTGATGTCGGTGCTCTTGAGAGTTTGAGGGGAAATGCAGATTTGGCTTAC
ATCCTGAGCATGGAGCCCTGTGGCCACTGCCTCATTATCAACAATGTGAACTTCTGC
CGTGAGTCCGGGCTCCGCACCCGCACTGGCTCCAACATCGACTGTGAGAAGTTGCG
GCGTCGCTTCTCCTCGCTGCATTTCATGGTGGAGGTGAAGGGCGACCTGACTGCCAA
GAAAATGGTGCTGGCTTTGCTGGAGCTGGCGCAGCAGGACCACGGTGCTCTGGACT
GCTGCGTGGTGGTCATTCTCTCTCACGGCTGTCAGGCCAGCCACCTGCAGTTCCCAG
GGGCTGTCTACGGCACAGATGGATGCCCTGTGTCGGTCGAGAAGATTGTGAACATCT
TCAATGGGACCAGCTGCCCCAGCCTGGGAGGGAAGCCCAAGCTCTTTTTCATCCAGG
CCTGTGGTGGGGAGCAGAAAGACCATGGGTTTGAGGTGGCCTCCACTTCCCCTGAA
GACGAGTCCCCTGGCAGTAACCCCGAGCCAGATGCCACCCCGTTCCAGGAAGGTTT
GAGGACCTTCGACCAGCTGGACGCCATATCTAGTTTGCCCACACCCAGTGACATCTT
TGTGTCCTACTCTACTTTCCCAGGTTTTGTTTCCTGGAGGGACCCCAAGAGTGGCTCC
TGGTACGTTGAGACCCTGGACGACATCTTTGAGCAGTGGGCTCACTCTGAAGACCTG
CAGTCCCTCCTGCTTAGGGTCGCTAATGCTGTTTCGGTGAAAGGGATTTATAAACAG
ATGCCTGGTTGCTTTAATTTCCTCCGGAAAAAACTTTTCTTTAAAACATCA
iCasp9 sequence (SEQ ID NO: 4):
ATGCTCGAGGGAGTGCAGGTGGAGACTATCTCCCCAGGAGACGGGCGCACCTTCCC
CAAGCGCGGCCAGACCTGCGTGGTGCACTACACCGGGATGCTTGAAGATGGAAAGA
AAGTTGATTCCTCCCGGGACAGAAACAAGCCCTTTAAGTTTATGCTAGGCAAGCAGG
AGGTGATCCGAGGCTGGGAAGAAGGGGTTGCCCAGATGAGTGTGGGTCAGAGAGCC
AAACTGACTATATCTCCAGATTATGCCTATGGTGCCACTGGGCACCCAGGCATCATC
CCACCACATGCCACTCTCGTCTTCGATGTGGAGCTTCTAAAACTGGAATCTGGCGGT
GGATCCGGAGTCGACGGATTTGGTGATGTCGGTGCTCTTGAGAGTTTGAGGGGAAAT
GCAGATTTGGCTTACATCCTGAGCATGGAGCCCTGTGGCCACTGCCTCATTATCAAC
AATGTGAACTTCTGCCGTGAGTCCGGGCTCCGCACCCGCACTGGCTCCAACATCGAC
TGTGAGAAGTTGCGGCGTCGCTTCTCCTCGCTGCATTTCATGGTGGAGGTGAAGGGC
GACCTGACTGCCAAGAAAATGGTGCTGGCTTTGCTGGAGCTGGCGCGGCAGGACCA
CGGTGCTCTGGACTGCTGCGTGGTGGTCATTCTCTCTCACGGCTGTCAGGCCAGCCA
CCTGCAGTTCCCAGGGGCTGTCTACGGCACAGATGGATGCCCTGTGTCGGTCGAGAA
GATTGTGAACATCTTCAATGGGACCAGCTGCCCCAGCCTGGGAGGGAAGCCCAAGC
TCTTTTTCATCCAGGCCTGTGGTGGGGAGCAGAAAGACCATGGGTTTGAGGTGGCCT
CCACTTCCCCTGAAGACGAGTCCCCTGGCAGTAACCCCGAGCCAGATGCCACCCCGT
TCCAGGAAGGTTTGAGGACCTTCGACCAGCTGGACGCCATATCTAGTTTGCCCACAC
CCAGTGACATCTTTGTGTCCTACTCTACTTTCCCAGGTTTTGTTTCCTGGAGGGACCC
CAAGAGTGGCTCCTGGTACGTTGAGACCCTGGACGACATCTTTGAGCAGTGGGCTCA
CTCTGAAGACCTGCAGTCCCTCCTGCTTAGGGTCGCTAATGCTGTTTCGGTGAAAGG
GATTTATAAACAGATGCCTGGTTGCTTTAATTTCCTCCGGAAAAAACTTTTCTTTAAA
ACATCAGTCGACTATCCGTACGACGTACCAGACTACGCACTCGAC
mCherry sequence (SEQ ID NO: 5)
ATGGTGAGCAAGGGCGAGGAGGATAACATGGCCATCATCAAGGAGTTCATGCGCTT
CAAGGTGCACATGGAGGGCTCCGTGAACGGCCACGAGTTCGAGATCGAGGGCGAGG
GCGAGGGCCGCCCCTACGAGGGCACCCAGACCGCCAAGCTGAAGGTGACCAAGGGT
GGCCCCCTGCCCTTCGCCTGGGACATCCTGTCCCCTCAGTTCATGTACGGCTCCAAG
GCCTACGTGAAGCACCCCGCCGACATCCCCGACTACTTGAAGCTGTCCTTCCCCGAG
GGCTTCAAGTGGGAGCGCGTGATGAACTTCGAGGACGGCGGCGTGGTGACCGTGAC
CCAGGACTCCTCCCTGCAGGACGGCGAGTTCATCTACAAGGTGAAGCTGCGCGGCA
CCAACTTCCCCTCCGACGGCCCCGTAATGCAGAAGAAGACCATGGGCTGGGAGGCC
TCCTCCGAGCGGATGTACCCCGAGGACGGCGCCCTGAAGGGCGAGATCAAGCAGAG
GCTGAAGCTGAAGGACGGCGGCCACTACGACGCTGAGGTCAAGACCACCTACAAGG
CCAAGAAGCCCGTGCAGCTGCCCGGCGCCTACAACGTCAACATCAAGTTGGACATC
ACCTCCCACAACGAGGACTACACCATCGTGGAACAGTACGAACGCGCCGAGGGCCG
CCACTCCACCGGCGGCATGGACGAGCTGTACAAGTAA
SEP sequence (SEQ ID NO: 6):
ATGAGTAAAGGAGAAGAACTTTTCACTGGAGTTGTCCCAATTCTTGTTGAATTAGAT
GGTGATGTTAATGGGCACAAATTTTCTGTCAGTGGAGAGGGTGAAGGTGATGCAAC
ATACGGAAAACTTACCCTTAAATTTATTTGCACTACTGGAAAACTACCTGTTCCTTG
GCCAACACTTGTCACTACTTTAACTTATGGTGTTCAATGCTTTTCAAGATACCCAGAT
CATATGAAACGGCATGACTTTTTCAAGAGTGCCATGCCCGAAGGTTATGTACAGGAA
AGAACTATATTTTTCAAAGATGACGGGAACTACAAGACACGTGCTGAAGTCAAGTTT
GAAGGTGATACCCTTGTTAATAGAATCGAGTTAAAAGGTATTGATTTTAAAGAAGAT
GGAAACATTCTTGGACACAAATTGGAATACAACTATAACGATCACCAGGTGTACAT
CATGGCAGACAAACAAAAGAATGGAATCAAAGCTAACTTCAAAATTAGACACAACA
TTGAAGATGGAGGCGTTCAACTAGCAGACCATTATCAACAAAATACTCCAATTGGC
GATGGGCCCGTCCTTTTACCAGACAACCATTACCTGTTTACAACTTCTACTCTTTCGA
AAGATCCCAACGAAAAGAGAGACCACATGGTCCTTCTTGAGTTTGTAACAGCTGCTG
GGATTACACATGGCATGGATGAACTATACAAA
CMV promoter sequence (SEQ ID NO: 7):
TAGTTATTAATAGTAATCAATTACGGGGTCATTAGTTCATAGCCCATATATGGAGTT
CCGCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTCCA
TTGACGTCAATGGGTGGAGTATTTACGGTAAACTGCCCACTTGGCAGTACATCAAGT
GTATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTACGT
ATTAGTCATCGCTATTACCATGGTGATGCGGTTTTGGCAGTACATCAATGGGCGTGG
ATAGCGGTTTGACTCACGGGGATTTCCAAGTCTCCACCCCATTGACGTCAATGGGAG
TTTGTTTTGGCACCAAAATCAACGGGACTTTCCAAAATGTCGTAACAACTCCGCCCC
ATTGACGCAAATGGGCGGTAGGCGTGTACGGTGGGAGGTCTATATAAGCAGAGCTG
GTTTAGTGAACCGTCAGATC
mPGK promoter sequence (SEQ ID NO: 8):
TTCTACCGGGTAGGGGAGGCGCTTTTCCCAAGGCAGTCTGGAGCATGCGCTTTAGCA
GCCCCGCTGGGCACTTGGCGCTACACAAGTGGCCTCTGGCCTCGCACACATTCCACA
TCCACCGGTAGGCGCCAACCGGCTCCGTTCTTTGGTGGCCCCTTCGCGCCACCTTCT
ACTCCTCCCCTAGTCAGGAAGTTCCCCCCCGCCCCGCAGCTCGCGTCGTGCAGGACG
TGACAAATGGAAGTAGCACGTCTCACTAGTCTCGTGCAGATGGACAGCACCGCTGA
GCAATGGAAGCGGGTAGGCCTTTGGGGCAGCGGCCAATAGCAGCTTTGCTCCTTCGC
TTTCTGGGCTCAGAGGCTGGGAAGGGGTGGGTCCGGGGGCGGGCTCAGGGGCGGGC
TCAGGGGGGGGCGGGCGCCCGAAGGTCCTCCGGAGGCCCGGCATTCTGCACGCTT
CAAAAGCGCACGTCTGCCGCGCTGTTCTCCTCTTCCTCATCTCCGGGCCTTTCGACCT
Linker between SEP and mCherry (GSSGSS linker) (SEQ ID NO 9):
GGCAGCAGCGGCAGCAGC
Cleavable P2A linker between iCaspase9 and SEP-mCherry (SEQ ID NO: 10):
GGAAGCGGAGCCACGAACTTCTCTCTGTTAAAGCAAGCAGGAGATGTTGAAGAAAA
CCCCGGGCCT
FKBP12-F36V Amino acid sequence (SEQ ID NO: 11):
GVQVETISPGDGRTFPKRGQTCVVHYTGMLEDGKKVDSSRDRNKPFKFMLGKQEVIRG
WEEGVAQMSVGQRAKLTISPDYAYGATGHPGIIPPHATLVFDVELLKLE
Truncated Caspase9 Amino acid sequence (AA135-416 from Homo sapiens Caspase9,
transcript variant alpha NM_001229.5) (SEQ ID NO: 12):
GFGDVGALESLRGNADLAYILSMEPCGHCLIINNVNFCRESGLRTRTGSNIDCEKLRRRF
SSLHFMVEVKGDLTAKKMVLALLELAQQDHGALDCCVVVILSHGCQASHLQFPGAVY
GTDGCPVSVEKIVNIFNGTSCPSLGGKPKLFFIQACGGEQKDHGFEVASTSPEDESPGSNP
EPDATPFQEGLRTFDQLDAISSLPTPSDIFVSYSTFPGFVSWRDPKSGSWYVETLDDIFEQ
WAHSEDLQSLLLRVANAVSVKGIYKQMPGCFNFLRKKLFFKTS
iCasp9 Amino acid sequence (SEQ ID NO: 13):
MLEGVQVETISPGDGRTFPKRGQTCVVHYTGMLEDGKKVDSSRDRNKPFKFMLGKQE
VIRGWEEGVAQMSVGQRAKLTISPDYAYGATGHPGIIPPHATLVFDVELLKLESGGGSG
VDGFGDVGALESLRGNADLAYILSMEPCGHCLIINNVNFCRESGLRTRTGSNIDCEKLRR
RFSSLHFMVEVKGDLTAKKMVLALLELARQDHGALDCCVVVILSHGCQASHLQFPGAV
YGTDGCPVSVEKIVNIFNGTSCPSLGGKPKLFFIQACGGEQKDHGFEVASTSPEDESPGSN
PEPDATPFQEGLRTFDQLDAISSLPTPSDIFVSYSTFPGFVSWRDPKSGSWYVETLDDIFE
QWAHSEDLQSLLLRVANAVSVKGIYKQMPGCFNFLRKKLFFKTSVDYPYDVPDYALD
mCherry Amino acid sequence (SEQ ID NO: 14)
MVSKGEEDNMAIIKEFMRFKVHMEGSVNGHEFEIEGEGEGRPYEGTQTAKLKVTKGGP
LPFAWDILSPQFMYGSKAYVKHPADIPDYLKLSFPEGFKWER VMNFEDGGVVTVTQDS
SLQDGEFIYKVKLRGTNFPSDGPVMQKKTMGWEASSERMYPEDGALKGEIKQRLKLKD
GGHYDAEVKTTYKAKKPVQLPGAYNVNIKLDITSHNEDYTIVEQYERAEGRHSTGGMD
ELYK
SEP Amino acid sequence (SEQ ID NO: 15):
MSKGEELFTGVVPILVELDGDVNGHKFSVSGEGEGDATYGKLTLKFICTTGKLPVPWPT
LVTTLTYGVQCFSRYPDHMKRHDFFKSAMPEGYVQERTIFFKDDGNYKTRAEVKFEGD
TLVNRIELKGIDFKEDGNILGHKLEYNYNDHQVYIMADKQKNGIKANFKIRHNIEDGGV
QLADHYQQNTPIGDGPVLLPDNHYLFTTSTLSKDPNEKRDHMVLLEFVTAAGITHGMDE
LYK
Amino acid sequence of Linker between SEP and mCherry (SEQ ID NO 16):
GSSGSS
Amino acid sequence of Cleavable P2A linker between iCaspase9 and SEP-mCherry
(SEQ ID NO: 17):
GSGATNFSLLKQAGDVEENPGP

1. A recombinant expression vector encoding an inducible cell death switch, a pH-stable fluorophore and a pH-sensitive fluorophore.

2. The recombinant expression vector according to claim 1, wherein the recombinant expression vector is a viral vector.

3. The recombinant expression vector according to claim 2, wherein the viral vector is a lentiviral vector.

4. The recombinant expression vector according to claim 1, wherein the inducible cell death switch induces apoptosis, necroptosis, pyroptosis or ferroptosis, preferably apoptosis.

5. The recombinant expression vector according to claim 1, wherein the inducible cell death switch comprises an inducer-binding domain and a signaling protein of a cell death pathway.

6. The recombinant expression vector according to claim 5, wherein the signaling protein of a cell death pathway is a pro-apoptotic protein, preferably Caspase9 or a functional fragment thereof.

7. The recombinant expression vector according to claim 5, wherein the inducer-binding domain comprises a dimerization domain, preferably FKBP12-F36V.

8. The recombinant expression vector according to claim 5, wherein the inducer-binding domain is capable of binding an inducer, preferably a chemical inducer of dimerization, most preferably AP20187.

9. The recombinant expression vector according to claim 1, wherein the inducible cell death switch is inducible Caspase 9.

10. The recombinant expression vector according to claim 1, wherein the pH-stable fluorophore and the pH-sensitive fluorophore have distinct excitation and emission spectra.

11. The recombinant expression vector according to claim 1, wherein the pH-stable fluorophore is from the RFP family, an Alexa Fluor dye, a protein-based fluorophore, a simple organic fluorophore or an organic polymer, preferably mCherry.

12. The recombinant expression vector according to claim 1, wherein the fluorescent signal of the pH-sensitive fluorophore changes in response to a decrease in pH, preferably wherein the fluorescent signal is quenched or the excitation/emission spectra are shifted.

13. The recombinant expression vector according to claim 1, wherein the pH-sensitive fluorophore is Superecliptic pHluorin (SEP), pHLemon, pHmScarlet, pHTomato, pHuji, a LysoSensor or a pH nanosensor, preferably Superecliptic pHluorin (SEP).

14. The recombinant expression vector according to claim 1, wherein the recombinant expression vector comprises a promoter, particularly an ubiquitous promoter, a cell specific promoter, a constitutive promoter, or an inducible promoter.

15. The recombinant expression vector according to claim 1, wherein the recombinant expression vector comprises a CMV promoter.

16. The recombinant expression vector according to claim 1, wherein the recombinant expression vector comprises a polynucleotide sequence according to SEQ ID NO: 1.

17. A cell comprising the recombinant expression vector according to claim 1.

18.-22. (canceled)

23. An in vitro method for evaluating phagocytosis, comprising the steps of:
a) providing an inducible substrate cell according to claim 17;
b) combining and co-culturing the inducible substrate cell with phagocytic cells;
c) inducing cell death of the inducible substrate cell; and
d) detecting the fluorescent signals of the inducible substrate cell;
wherein a change in fluorescent signal of the pH-sensitive fluorophore is indicative of phagocytosis of the inducible substrate cell.

24. The method according to claim 23, wherein the phagocytic cells of step b) are macrophages or tissue resident macrophages.

25. The method according to claim 24, wherein the tissue resident macrophages are microglia.

26. The method according to claim 23, wherein the inducible substrate cell and the phagocytic cells are co-cultured in an in vitro model comprising additional cell types.

27. The method according to claim 26, wherein the in vitro model is a 2D or 3D culture system, an organ-on-a-chip, a spheroid, or an organoid.

28. The method according to claim 26, wherein the in vitro model is an organ-on-a-chip, spheroid, an organoid, a neurovascular unit, or a blood brain barrier spheroid.

29. (canceled)

30. The method according to claim 23, wherein cell death of the inducible substrate cell in step c) is induced after the inducible substrate cell is combined with the phagocytic cells in step b).

31. The method according to claim 23, wherein cell death of the inducible substrate cell in step c) is induced by an inducer, preferably a chemical inducer of dimerization, most preferably AP20187.

32. The method according to claim 23, wherein the fluorescent signals of step d) are detected by fluorescent imaging, flow cytometry or by a fluorescence plate reader.

33. The method according to claim 23, wherein the fluorescent signal in step d) is detected at several time points after induction of cell death.
